(19) United States
(12) Patent Application Publication
(10) Pub. No.: US 2013/0172961 A1
(43) Pub. Date: Jul. 4, 2013

(54) Flash lamp method for treating onychomycosis

(71) Applicant: Jan Enemaerke, San Marcos, CA (US)

(72) Inventor: Jan Enemaerke, San Marcos, CA (US)

(21) Appl. No.: 13/374,662

(22) Filed: Jan. 4, 2012

(51) Int. Cl. A61N 5/06

(52) U.S. Cl. 607/90

(57) ABSTRACT

A rechargeable battery operated hand-held light emitting device that will treat the symptoms of common onychomycosis. The device preferably is operated utilizing a flash lamp of as the source of energy, a reflector and special window filter limiting the wavelength output to the narrow band range of 900-1100 nm. This will optimize the absorption of light energy in the nail bed, and thus increase the temperature of the nail bed to a efficacious level, by and while transmitting the optical light energy through the nail. The targeted tissue is located below the nail and is heated by a series of flashes in a sequential release of energy. A disposable treatment tip can be attached to the hand-held device to ensure safety in form of tissue contact and providing user safety.

### FIELD OF THE INVENTION

The present invention relates to medical device and especially to optical devices for treatment of skin diseases.

### BACKGROUND OF THE INVENTION

Onychomycosis (also known as “dermatophytic onychomycosis, ringworm of the nail) is a type of fungus and is the most common disease of the nails and constitutes about a half of all nail abnormalities. This condition may affect toenails or fingernails, but toenail infections are particularly common. The nail plate can have a thickened, yellow, or cloudy appearance. The nails can become rough and crumbly, or can separate from the nail bed. There is usually no pain or other bodily symptoms, unless the disease is severe. Patients with onychomycosis may experience significant psychosocial reactions.

Risk factors for onychomycosis include family history, increasing age, poor health, prior trauma, warm climate, participation in fitness activities, immunosuppression (e.g., HIV, drug induced), communal bathing, and occlusive footwear. Treatment of onychomycosis is challenging because the infection is embedded within the nail and is difficult to reach. As a result full removal of symptoms is very slow and may take a year or more. Most treatments are either systemic antifungal medications such as terbinafine and itraconazole, or topical such as nail paints containing ciclopirox or amorolfine. There is also evidence for combining systemic and topical treatment.

Nail lacquer (with consistent application) may be used as a method of removing the fungus in mild to moderately infected nails only. The lacquer must be coated onto the infected nail(s) and surrounding skin daily for seven days. Following the seven day coating period, the layered lacquer must be wiped off the nails, and one must begin the process once again. This procedure must be consistently repeated for at least one year until the nail infections have cleared up. This process lacks effectiveness as research has shown that it cured less than 10 percent of infected people using it. Additionally, if one's infection is severe or extremely painful, it is advised for one to remove their infected nail as to allow a new one to grow in its place. This method is time consuming and an effective recovery is not guaranteed.

Other methods include oral medication, such as: Itraconazole (Sporanox), Fluconazole (Diflucan), or Terbinafine (Lamisil). These medications permit the nail to grow infection-free, gradually replacing the infected part of one's nail. These medications must be consumed for six to twelve weeks, but will not see the final result until the nail fully grows out. However, it may take four months or longer to eliminate an infection, depending on the severity of the infection, as well as the rate at which one's nails grow. Future infections are likely to occur, principally if one continues to expose the nails to warm and/or moist conditions. Drawbacks of this method include side effects such as: headache, upset stomach, skin rashes, or allergic reactions to ingredients in the medication. Other side effects (of a more serious nature) include: liver damage and heart failure. Amorolfine is currently the most effective topical treatment for onychomycosis, but is not approved in the United States or Canada. Amorolfine 5% nail lacquer in once-weekly or twice-weekly applications has been shown in well designed, placebo controlled studies to be between 60% and 71% effective in treating toenail onychomycosis; complete cure rates three months after stopping treatment (after six months of treatment) is estimated to be between 38% and 46%. Itraconazole is effective for oral treatment of onychomycosis. Pulse dosing (escalating drug levels early in the dosing interval followed by a prolonged dose-free period) is 61% to 75% effective in providing a complete cure.

Lasers are being used for treatment of toenail fungus. These lasers use Nd:YAG lasers to target the fungus while leaving surrounding nail and tissue undamaged. The PinPointe footlaser has been cleared by the FDA for use in onychomycisis and is available throughout the USA, UK, Germany, Turkey and Australia. Published research has shown an effectiveness of between 70-80% and in many cases a single treatment is sufficient. This treatment is safe and has no side effects since the laser is applied directly to the infection.

A Noveon-type laser that is already in use by physicians for some types of cataract surgery is used by some podiatrists although the only scientific study on its efficacy, while showing positive results, included far too few test subjects for the laser to be proven generally effective.

The Ontario Osteopathic and Alternative Medicine Association in Ontario, Canada, have developed a laser-based method, “LAFT” which is claimed to have a 96% success rate based on “hundreds of treatments given”. To date there are several lasers seeking approval and one that has been cleared by the Food and Drug Administration. Lasers typically are expensive and most are bulky and typically are designed for use in medical facilities.

Use of flash lamps for treatment of skin conditions has been proposed. For example, see U.S. patent application Ser. No. 12/393,995 describing techniques for treatment of acne in which flash lamp energy is used to kill the bacteria that causes acne. This patent application including the detailed electronic design of the flash lamp is hereby incorporated herein by reference.

What is needed is an inexpensive hand held device for self treatment at home of onychomycosis.

### SUMMARY OF THE INVENTION

The present invention provides a rechargeable battery operated hand-held light emitting device that will treat the symptoms of common onychomycosis. The device preferably is operated utilizing a flash lamp of as the source of energy, a reflector and special window filter limiting the wavelength output blocking ultraviolet. This will optimize the absorption of light energy in the nail bed, and thus increase the temperature of the nail bed to an efficacious level, by and while transmitting the optical light energy through the nail. The targeted tissue is located below the nail and is heated by a series of flashes in a sequential release of energy. A disposable treatment tip can be attached to the hand-held device to ensure safety in form of tissue contact and providing user safety.

### BRIEF DESCRIPTION OF THE DRAWINGS

FIG. 1 is a sketch of a preferred flash lamp unit utilized in preferred embodiments.

FIG. 2 shows a set of cone delivery tips.

FIG. 3 shows specifications for a preferred filter unit to provide a preferred spectral distribution of flash lamp energy.

### DETAILED DESCRIPTION OF PREFERRED EMBODIMENTS

There is a significant demand for devices that can treat skin conditions, in particular for those that treat Onychomycosis. Onychomycosis affects the nail bed of both fingers and toes and it can affect people of all ages. As indicated in the background section several types of treatments for Onychomycosis are known, including topical antibiotics, oral antibiotics, and prescription drugs. All have either very low efficacy or undesirable side effects. Devices including lasers have also been used to treat Onychomycosis, but the equipment is often large and difficult to use.

### First Preferred Embodiment

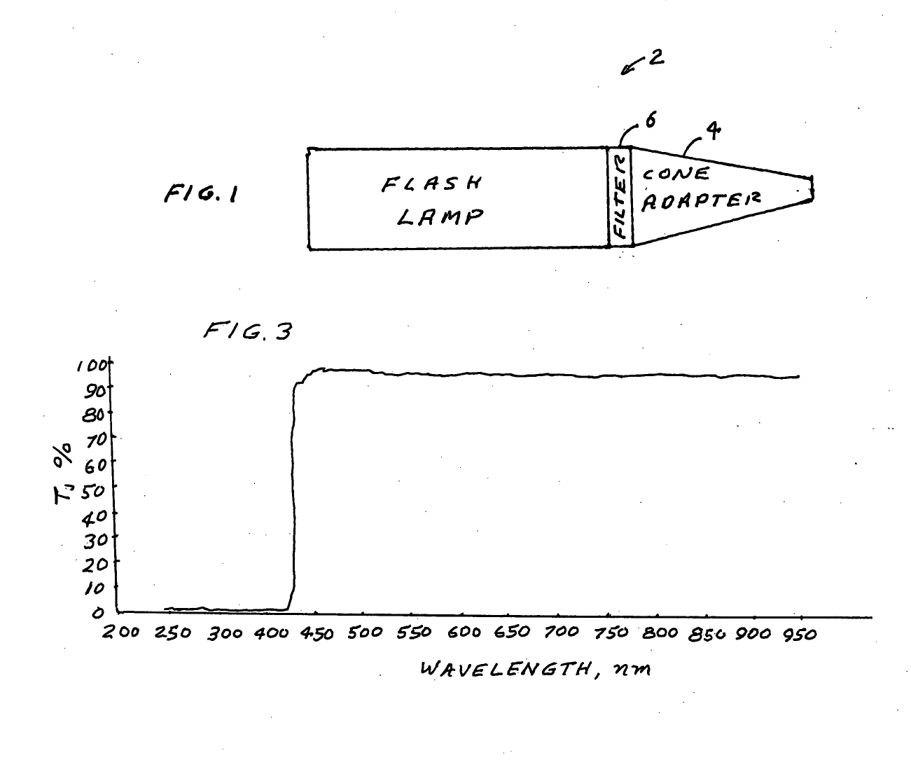

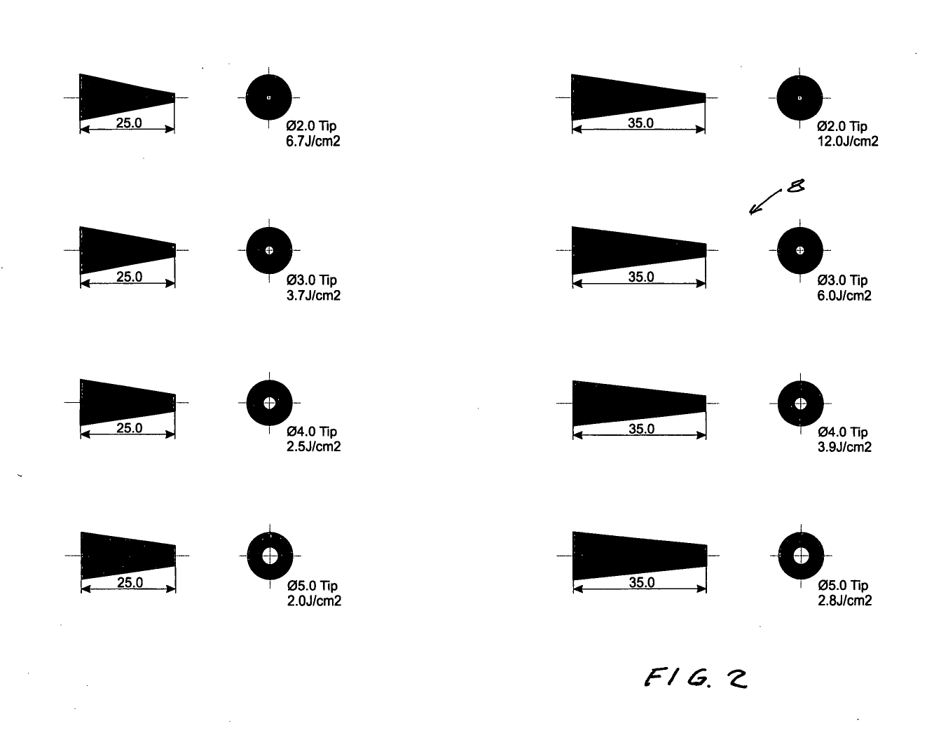

In a first preferred embodiment a method is provided for maintaining a therapeutically significant level of energy at a treatment area utilizing a series of multiple flashes delivered to the treatment area by a hand held xenon flash lamp sketched at 2 in FIG. 1. The output of the flash lamp is filtered with a special filter to block ultraviolet and deliver mostly infrared energy to the treatment area via a cone shaped optical delivery tip. The cone shaped delivery tip 4 is preferably one of the cones shown in FIG. 2. Applicant's choice of these cones is the cone shown at 8 in FIG. 2 with an exit diameter of 3 mm (an area of 0.706 cm²). Preferably the cone tip is placed adjacent to the treatment area to limit the spread of the infrared energy. The preferred flash lamp produces about 0.5 joules per pulse, at a pulse frequency of 2 pulses per second and the preferred treatment time is 6 seconds for a total energy per treatment of 6 joules. However much of that energy is lost in the filter and the cone delivery tip and much of the energy delivered to the tissue is dissipated during the 6 seconds of treatment so that the total heat energy delivered to the treatment area is only about 6 J/cm² which increases the temperature in the treatment area to a temperature of somewhat less than 42° C. which is sufficient to kill the onychomycosis fungus with no significant and no permanent damage to tissue in the treatment area. At 42° C. the patient will feel warmth in the treatment area but no significant pain. The treatments should be repeated so as to include all tissue containing fungal infection. Preferably the procedure can be repeated daily for about seven days or until no sign of infection exists.

### Flash Lamp Design

The flash lamp, as indicated above, is a xenon flash lamp designed to produce ultraviolet, visible and infrared light. The flash lamp may be an off-the-shelf flash lamp such as Model FF-O-1016001340 supplied by Nam Kwong Electric Cl Ltd with offices in Hong Kong, China. Or the lamp may be a lamp similar to the one described in patent application Ser. No. 12/393,995 which is referrerd to in the background section and has been incorporated herein by reference. The filter shown at 6 in FIG. 1 is designed to block substantially all ultraviolet light. Enough visible light is transmitted so that the operator can see light exiting the cone. A preferred embodiment utilizes a filter supplied by LOHNSTAT Coating with offices in Escondido, California. This filter produces a spectral performance as indicated in FIG. 3. The flash lamp unit includes an energy storage device, preferably a capacitor which is charged prior to each pulse and a controller configured to flash the flash lamp, by delivering energy from said energy storage device to said flash lamp. Pulse energies, pulse repletion rates and beam size preferably may be as specified above, but other combinations could be utilized with results just as good. The flashes should be repeated a sufficient number of times within a predetermined treatment period to raise the temperature of a treatment area having a fungal infection, onychomycosis, to about 42 degrees Celsius during said treatment period. The sufficient number of times will normally be twelve, or less, for example. The predetermined treatment period would normally be less than can be about 14 seconds depending on the chosen repetition rates and beam size.

As described in the reference device the flash lamp includes a user input in some embodiments wherein said predetermined therapeutic level is determined on said user input. The device may be configured to provide a predetermined therapeutic level based upon user input. The device also comprises a controller discreet logic in some embodiments. Preferred embodiments include the following features:
Hand held—multi-flash flash light based device for treatment of onychomycosis,
Filter window cut offs
Conical treatment tip
Energy levels up to 6 J/cm² delivered to the treatment areas

### Variations

While the present invention has been described in terms of preferred embodiments the reader will recognize that many changes and modifications can be made within the general scope of the present invention. For example, many other combinations of pulse repetition rates, pulse energies and treatment areas can be substituted for the preferred combination described to provide heat energy delivered to the treatment area of about 6 J/cm². For example the pulse rate could be doubled and the time period halved. The pulse energy could be increased and the treatment area correspondingly expanded. For some people a somewhat greater or lesser heat rate may be required; however, Applicant recommends the total heat energy delivered to the treatment area of between 4 J/cm² to 8 J/cm².

Therefore the scope of the present invention should be determined by the claims and their legal equivalence.

What is claimed is:

1. A method to treat and cure onychomycosis caused by infecting fungi comprising the steps of:
A) providing a hand held broadband pulsed light source with output wavelengths primarily in the visible and infrared spectral range;
B) administering a pre-set dosage of broadband pulsed light with a total energy density of approximately 4 to 8 joules/cm²;
wherein the broad band pulsed light heats the target tissue to a sufficient temperature to damage infecting fungi, but not cause any permanent damage to the patient's tissues.

2. The method of claim 1 wherein the broadband pulsed light source is a xenon flash lamp.

3. The method of claim 1 wherein the broadband light comprises a filter adapted to limit the frequency of the broadband light primarily to between 900 nm and 1100 nm.

4. The method of claim 1 wherein the broadband light source comprises a conical tip.

5. The method of claim 1 wherein the broadband light source is controlled by use of a controller.

6. The method of claim 4 where the broadband light source comprises a safety shield feature adapted to prevent the discharge of light into open space unless the conical tip is in contact with the treatment area of the patient.

7. A method to treat and cure onychomycosis caused by Candida or similar yeasts in a patient comprising the steps of:
A) using a hand held pulsed light from a broadband light source with output wavelengths of approximately 900 nm-1100 nm;
B) administering a pre-set dosage of light with a total energy density of approximately 6 joules/cm², wherein the broad band pulsed light heats the target tissue to a sufficient temperature to damage Candida or similar yeasts, but not cause any permanent damage to the patient's tissues.

8. The method of claim 7 where the broadband light is produced by at least one xenon flash lamp.

9. The method of claim 7 where the broadband light is filtered using cutoff filters to minimize light with wavelengths below 900 nm and above 1100 nm from reaching the target tissue.

10. The method of claim 7 where the light is delivered via a conical fiber optic tip.

11. The method of claim 7 where the dosage of light and number of light flashes is controlled by use of a controller.

12. The method of claim 7 wherein the light source comprises a safety feature to prevent the discharge of light into open space and thus preventing ocular damage.

13. A method to treat and cure onychomycosis caused by nondermatophytic molds in a patient by comprising the steps of:
A) using a hand held pulsed light from a broadband light source with output wavelengths of approximately 900 nm-1100 nm;
B) administering a pre-set dosage of light with a total energy density of approximately 6 joules/cm²,
wherein the broad band pulsed light heats the target tissue to a sufficient temperature to damage the nondermatophytic molds but not cause any permanent damage to the patient's tissues.

14. The method of claim 13 where the broadband light is produced by at least one xenon flash lamp.

15. The method of claim 13 where the broadband light is filtered using cutoff filters to minimize light with wavelengths below 900 nm and above 1100 nm from reaching the target tissue.

16. The method of claim 13 where the light is delivered via a conical fiber optic tip.

17. The method of claim 13 where the dosage of light and number of light flashes is controlled by use of a controller.

18. The method of claim 13 wherein the light source comprises a safety feature to prevent the discharge of light into open space and thus preventing ocular damage.
